(19) United States
(12) Patent Application Publication
(10) Pub. No.: US 2017/0042502 A1
(43) Pub. Date: Feb. 16, 2017

(54) Lung Phantom System For 4D-CT

(71) Applicant: Research & Business Foundation SUNGKYUNKWAN UNIVERSITY, Suwon-si (KR)

(72) Inventors: Ja Choon KOO, Seoul (KR); Hyuk Jin LEE, Changwon-si (KR); Hae Jin LEE, Incheon (KR); Ji Na KIM, Seoul (KR); Young Nam KANG, Seoul (KR); Hong Seok JANG, Seoul (KR)

(73) Assignee: Research & Business Foundation SUNGKYUNKWAN UNIVERSITY, Suwon-si (KR)

(21) Appl. No.: 15/234,261

(22) Filed: Aug. 11, 2016

(30) Foreign Application Priority Data
Aug. 12, 2015 (KR) 10-2015-0114084

(51) Int. Cl. A61B 6/00; G09B 23/32; A61B 6/03

(52) U.S. Cl. CPC A61B 6/583; A61B 6/032; G09B 23/32

(57) ABSTRACT

The present disclosure relates to a lung phantom system provided with an elastic film that separates a first chamber and a second chamber having inner space that may be filled with liquid, the elastic film configured to repeat expansion and restoration according to introduction and discharge of liquid, thereby providing an effect of accurately replicating movements of an actual lung during its respiratory movement.

### CROSS-REFERENCE TO RELATED APPLICATION

This application claims priority to and the benefit of Korean Patent Application No. 10-2015-0114084, filed on Aug. 12, 2015, in the Korean Intellectual Property office, the entire contents of which are incorporated herein by reference in their entirety.

### BACKGROUND

Field

The present disclosure relates to a phantom system. More particularly, the present disclosure relates to a lung phantom system capable of replicating an actual respiratory movement with accuracy.

Description of Related Art

Due to changes in life patterns and eating habits caused by modernization, incidence rates of various kinds of cancer are increasing. In addition, medical equipment has advanced and so it has become possible to photograph a human body and diagnose cancer at an early stage using various newly developed diagnostic devices, identify the location of cancer cells, and kill or prevent the cancer cells from propagating further using radiation treatment and the like, thereby treating the cancer or alleviating the pain of the patient.

However, exposing an actual human body to a diagnostic device in order to check the accuracy, performance and the like of the device in the process of developing and manufacturing the new diagnostic device is very dangerous.

Therefore, when developing various kinds of medical equipment or treatment methods, a phantom, which is a model of a human body, is used.

There are two kinds of such phantoms: static model phantoms and dynamic model phantoms.

Static models are phantom models used when taking X-rays, 3D-CT and the like. These models replicate only the shapes of a human body, and not its movement.

Dynamic models are phantom models made to replicate not only the shapes of a human body such as a heart or lung, but also its movements.

However, in conventional phantom systems, water is thrusted into a sponge phantom having the shape of a human body and then the phantom is contracted to replicate movements of the human body, and thus pressure is applied evenly to all surfaces of the phantom, making all the surfaces of the phantom contract or expand identically.

Otherwise, one surface of the phantom is thrusted perpendicularly so that it contracts linearly.

However, such movements are quite different from the movements of an actual lung, and thus there is a need to develop a lung phantom system that can replicate the actual respiratory movements with accuracy.

### SUMMARY

A purpose of the present disclosure is to solve the aforementioned problems of prior art, that is, to provide to a lung phantom system capable of replicating respiratory movements of an actual lung with accuracy.

Another purpose of the present disclosure is to provide a lung phantom system capable of replicating movements of an actual lung with accuracy by using an elastic film.

Another purpose of the present disclosure is to provide a lung phantom system capable of accurately replicating movements of air being inhaled and exhaled during an actual respiration by using sine waveform driving signals.

Another purpose of the present disclosure is to provide a lung phantom system capable of replicating movements of an actual lung with accuracy by using a phantom formed by two or more kinds of blocks having different elastic moduli.

Another purpose of the present disclosure is to provide a lung phantom system capable of representing target cells such as cancer cells by forming a phantom including a target block displayed differently from the remaining blocks in a photographed image of a diagnostic device.

According to an embodiment of the present disclosure, there is provided a lung phantom system including a first chamber and a second chamber having inner space that may be filled with liquid, the first chamber and the second chamber separated by an elastic film; and a liquid supply unit that supplies the liquid into the second chamber through a liquid inlet formed in the second chamber or removes the liquid from the second chamber through the liquid inlet, wherein the elastic film expands towards inside the first chamber as the liquid is supplied by the liquid supply unit into the second chamber, and the elastic film is restored back to its original position as the liquid is discharged from the second chamber by the liquid supply unit.

The liquid supply unit may include a liquid supply tube connected with the liquid inlet; a liquid supplier that supplies the liquid to the liquid supply tube or sucks the liquid from the liquid supply tube; and a driver that actuates the liquid supplier.

A driving signal of the driver may be a signal having a sine waveform replicating respiration.

The lung phantom system may further include a phantom that is disposed inside the first chamber, and that contracts as the elastic film expands and is restored back to its original state as the elastic film is restored.

The lung phantom system may further include an air tube formed to penetrate one surface of the first chamber so as to connect the inner space and outer space of the first chamber; and an inner film that surrounds the phantom such that the liquid does not directly contact the phantom but such that the phantom and outside air are communicable with each other through the air tube.

The phantom may consist of at least two groups of blocks having different elastic moduli from each other.

The phantom may consist of a plurality of layers, and a plurality of the blocks forming at least one layer of the plurality of layers may consist of the at least two groups of blocks having different elastic moduli from each other.

A portion of the blocks may be a target block that is displayed differently from the other blocks when photographing the phantom with a diagnostic device.

The present disclosure can provide a lung phantom system capable of accurately replicating movements of an actual lung in respiratory movements. Further, such a phantom can represent movements identical to the actual movements and locations of a tumor, thereby providing an effect as an alternative to patients when assessing 4D-CT performance and measuring the amount of radiation, etc.

### BRIEF DESCRIPTION OF THE DRAWINGS

Example embodiments will now be described more fully hereinafter with reference to the accompanying drawings; however, they may be embodied in different forms and should not be construed as limited to the embodiments set forth herein. Rather, these embodiments are provided so that this disclosure will be thorough and complete, and will fully convey the scope of the example embodiments to those skilled in the art.

In the drawing figures, dimensions may be exaggerated for clarity of illustration. It will be understood that when an element is referred to as being “between” two elements, it can be the only element between the two elements, or one or more intervening elements may also be present between two elements. Like reference numerals refer to like elements throughout.

FIG. 1 is a schematic view of a lung phantom system according to an embodiment of the present disclosure;

FIG. 2 is a view illustrating movements of a lung phantom system provided to illustrate the shape of a lung during an exhale;

FIG. 3 is a view illustrating movements of a lung phantom system provided to illustrate the shape of a lung during an inhale;

FIG. 4 is a schematic view of a lung phantom system according to another embodiment of the present disclosure;

FIGS. 5 and 6 are perspective views exemplifying a phantom structure that may be used in a lung phantom system according to the present disclosure;

FIG. 7 is a view exemplifying a screen of a phantom including a target block, photographed by a diagnostic device;

FIG. 8A is a photograph showing the shape of a lung phantom system during an inhale according to an embodiment of the present disclosure;

FIG. 8B is a photograph showing the shape of a lung phantom system during an exhale according to an embodiment of the present disclosure;

FIG. 9A is a photograph showing the shape of an actual lung during an inhale; and

FIG. 9B is a photograph showing the shape of an actual lung during an exhale.

### DETAILED DESCRIPTION

Hereinafter, a lung phantom system according to the present disclosure will be explained in detail with reference to the drawings attached.

In the explanation hereinafter, only the portions necessary for understanding a lung phantom system according to embodiments of the present disclosure will be explained, and explanation on other portions may be omitted so as not to obscure the main point of the present disclosure.

Further, the terms and words used in the present specification and claims to be explained hereinafter should not be construed as limited to their general or dictionary meanings, but should be construed as meanings and concepts suitable to the technical concept of the present disclosure that express the present disclosure most suitably.

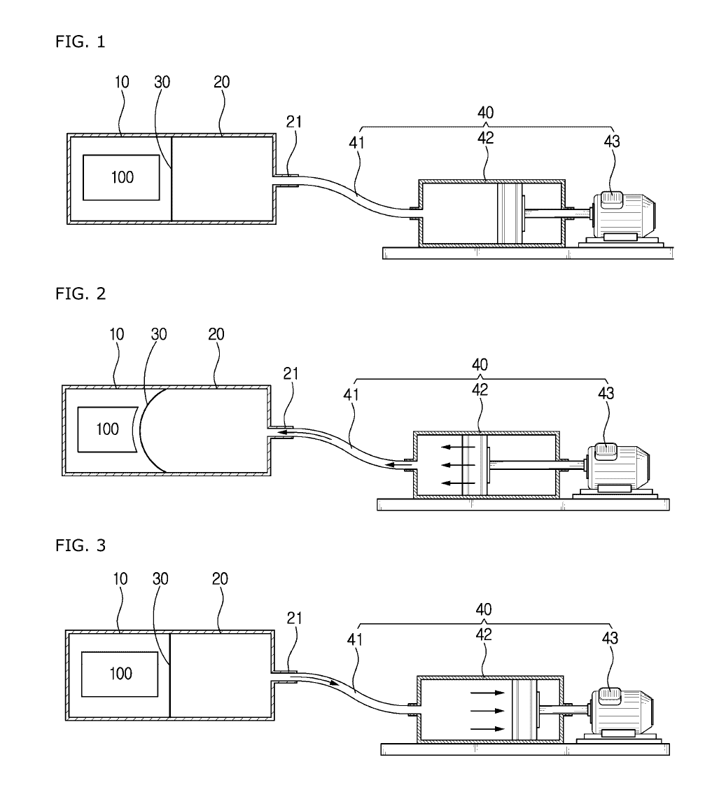

FIG. 1 is a schematic view illustrating a lung phantom system according to an embodiment of the present disclosure.

The lung phantom system according to the present disclosure includes a first chamber 10, a second chamber 20, an elastic film 30 and a liquid supply unit 40, as illustrated in FIG. 1.

More specifically, the first chamber 10 and the second chamber 20 are chambers having inner space that may be filled with liquid.

The liquid filling the first chamber and the second chamber may be liquid such as water.

The elastic film 30 is disposed between the first chamber and the second chamber, and the liquid inside the first chamber and the liquid inside the second chamber are separated by the elastic film 30 and thus do not mix.

As illustrated in FIG. 1, a phantom 100 is disposed inside the first chamber.

Inside the second chamber 20, a liquid inlet 21 is provided. By the liquid supply unit 40 connected to the liquid inlet 21, liquid may be supplied to or removed from the inside of the second chamber.

In the case where liquid is supplied by the liquid supply unit 40 even after the second chamber 20 is filled with liquid, as illustrated in FIG. 2, the elastic film 30 expands towards the inside of the first chamber 10 due to pressure, whereby the inner space of the first chamber decreases, and by this pressure, the phantom 100 contracts.

Further, in the case where liquid is removed by the liquid supply unit 40, as illustrated in FIG. 3, the elastic film 30 is returned back to its original position, whereby the inner space of the first chamber increases again, and the phantom 100 expands again and is returned back to its original shape.

It is desirable that the elastic film 30 is made of a material having elastic force in a shape that can form a flat diaphragm between the first chamber 10 and the second chamber 20 when pressure is not applied so that it operates to replicate the respiratory movements mentioned above.

Further, it is desirable that the elastic film 20 has as much elastic force and rigidity as not to be deformed or damaged even after repeating the operations of replicating the respiratory movements.

As illustrated in FIG. 1, the liquid supply unit 40 that supplies liquid to the inside of the second chamber 20 may include a liquid supply tube 41 connected to the liquid inlet 21 of the second chamber, a liquid supplier 42 that supplies or discharges liquid to the liquid supply tube 41, and a driver 43 that actuates the liquid supplier 42.

More specifically, the liquid supply tube 41 is connected between the second chamber 20 and the liquid supplier 42 to provide a space where the liquid can move. The liquid supplier 42 can be directly connected to the second chamber. However, by spacing the liquid supplier 42 that includes a metal material apart from the second chamber using a liquid supply tube made of a non-metal material, it is possible to prevent the metal artifact phenomenon that may occur during a computerized tomography (CT).

Further, the liquid supply tube 41 also has an effect of preventing damage to the chamber unlike in a conventional phantom system where the liquid supplier 42 is directly connected to a chamber.

The liquid supplier 42 is a device configured to supply or discharge liquid. As illustrated in FIG. 1, the liquid supplier 42 may be a piston device, a pump or the like, but there is no limitation thereto, and thus any device configured to supply and discharge liquid may be used as the liquid supplier 42.

The liquid supplier is actuated by the driver 43 such as an actuator, wherein it is desirable that a signal of the driver 43 is a signal having a sine waveform so as to similarly replicate respiration of an actual lung.

Further, in the case where the lung phantom system replicates lung movements of a certain patient, it is desirable that the respiration volume during an exhale and an inhale of the patient are measured, and that a driving signal of the liquid driver is a signal having the sine waveform corresponding to the measured respiration volume.

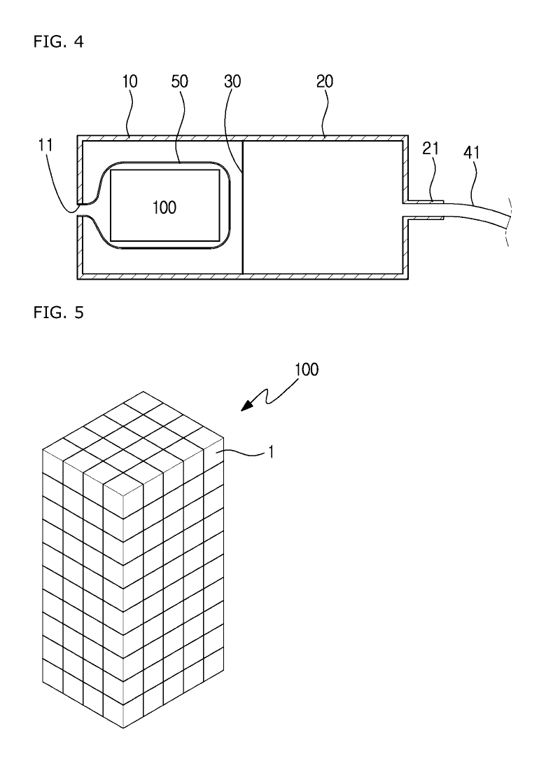

FIG. 4 illustrates a lung phantom system according to another embodiment of the present disclosure. Unlike the lung phantom system of FIG. 1, the lung phantom system according to the another embodiment of the present disclosure illustrated in FIG. 4 has an air tube 11 formed inside the first chamber 10, and further includes an inner film 50 that surrounds the phantom 100 disposed inside the first chamber.

More specifically, the air tube 11 is formed to penetrate one surface of the first chamber so as to connect the inner space and the outer space of the first chamber.

The inner film 50 is a thin film like a plastic bag that surrounds the phantom to prevent the liquid inside the first chamber from directly contacting the phantom 100 as illustrated in FIG. 4, so that the phantom 100 is communicable with outside air through the air tube.

In such a lung phantom system according to the another embodiment of the present disclosure, when liquid flows inside the second chamber 20 and thus the elastic film 30 expands towards the inside of the first chamber 10, the pressure being applied to the phantom increases, whereby the air that used to be inside the phantom is discharged towards outside through the air tube 11 and the volume of phantom 100 contracts.

Then, when the liquid is discharged from the inside of the second chamber 20 and thus the elastic film 30 returns back to its original position, the pressure being applied to the phantom decreases, whereby outside air naturally flows into the inner film 50 through the air tube 11 and the volume of the phantom also increases to its original shape.

Such a lung phantom system according to the another embodiment of the present disclosure may provide movements of the phantom that are similar to the respiratory movements of an actual lung, as the air inside the phantom is naturally discharged towards outside and then introduced back in.

In such a lung phantom system according to the embodiments of the present disclosure explained hereinabove, the phantom 100 may be made of a material having elasticity and compressibility such as sponge, latex and the like so that it may be restored back to its original state when pressure is removed.

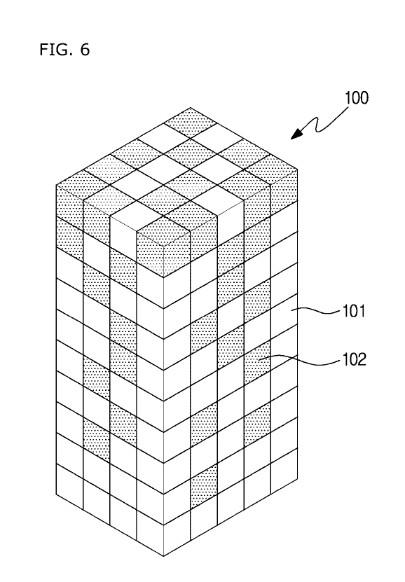

Especially, by attaching a plurality of unit blocks 1 to form a lung phantom as illustrated in FIG. 5, it is possible to easily form a lung phantom replicating different shapes and sizes for different patients.

Especially, since a lung does not contract or expand identically in every position during an exhale and an inhale, it is desirable to form a phantom using at least two kinds of blocks 101, 102 having different moduli from each other as illustrated in FIG. 6 so that the lung movements can be replicated with accuracy.

More specifically, a phantom may be formed using at least two groups of blocks 101, 102 as illustrated in FIG. 6. If the elastic modulus of a B group block 102 is higher than that of an A group block 101, the B group block 102 is harder than the A group block, and thus the B group block 102 contracts less than the A group block under a same pressure, and when the pressure is increased or decreased, the sponge block in the A group 101 contracts or expands further than the blocks in the B group.

By using blocks having different elastic moduli as aforementioned, it is possible to easily form a phantom replicating a human organ of which the composition differs from person to person.

Further, by arranging a greater number of blocks with low elastic modulus in the lower portion of the phantom 100 than in the upper portion thereof as illustrated in FIG. 6, it is possible to replicate lower areas of the lung where the volume change rate is higher than upper areas according to respiratory movements with increased accuracy.

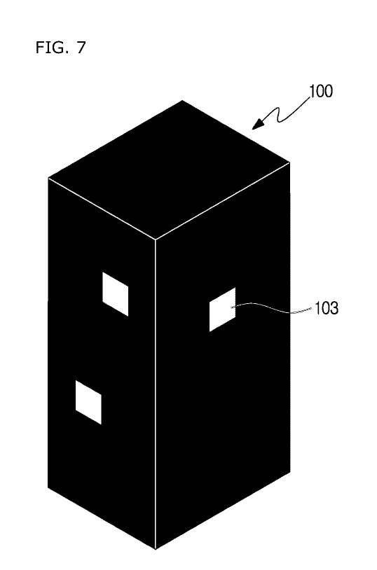

Further, in the case of photographing some of the blocks forming a lung phantom with a diagnostic device such as an ultrasound device, CT and the like as illustrated in FIG. 7, by forming a target block 103 to be displayed differently from the rest of the blocks in a photographed image, it becomes possible to see how a target cell such as a cancer cell in the lung moves during respiratory movements.

For this purpose, it is desirable that the target block 103 is made of a material having a density different from the rest of the blocks. Here, it is desirable that the density is similar to that of human skin, muscles, and organs; lower than that of human bone; higher than that of water; and lower than that of the chamber.

Further, since the target block 103 should be able to change its shape together with the other blocks according to respiratory movements, it is desirable that the target block 103 is made of a material having elastic force.

Therefore, the target block 103 may be made of silicon rubber or a material similar to silicon rubber having high elasticity and tensile force, and suitable density.

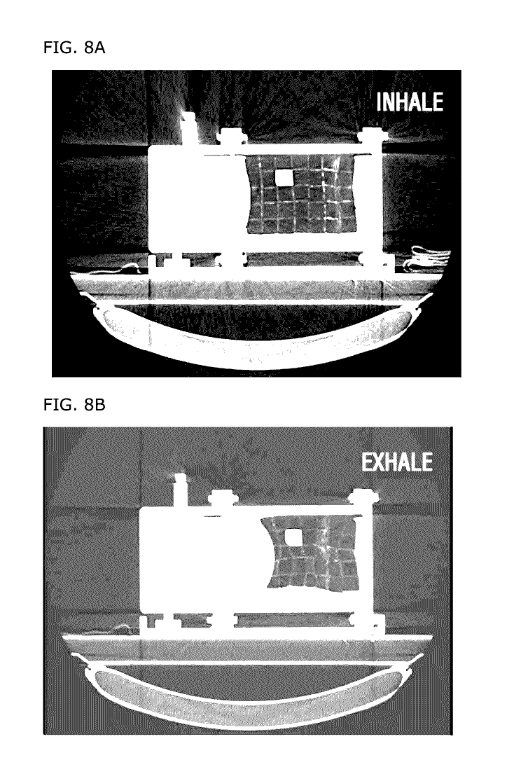

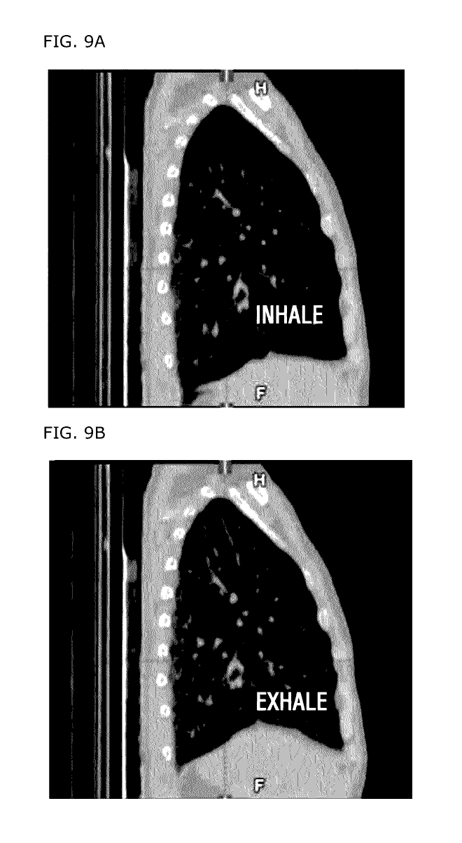

CT photographs taken while operating a lung phantom system according to the embodiment of the present disclosure explained hereinabove are illustrated in FIG. 8A and FIG. 8B, and one can see that the shapes of the phantom of FIG. 8A and FIG. 8B are very similar to the photographs of an actual lung illustrated in FIGS. 9A and 9B.

Further, since it is possible to see movements of the target block 103 as well, it is possible to accurately replicate movements of a tissue such as a lung that has high volume change rates and possibilities that the location, shape and the like of a tumor may change depending on movements, proving that the lung phantom system according to the embodiments of the present disclosure may be used as an alternative to patients when assessing 4D-CT performance and measuring the amount of radiation, etc.

So far, a phantom and a lung phantom system according to the present disclosure were explained with reference to specification embodiments of the present disclosure.

In the drawings and specification, there have been disclosed typical embodiments of the invention, and although specific terms are employed, they are used in a generic and descriptive sense only and not for purposes of limitation. It will be understood by those of ordinary skill in the art that various changes in form and details may be made therein without departing from the spirit and scope of the present invention as defined by the following claims.

What is claimed is:

1. A lung phantom system comprising:
a first chamber and a second chamber having inner space that may be filled with liquid, the first chamber and the second chamber separated by an elastic film; and
a liquid supply unit that supplies the liquid into the second chamber through a liquid inlet formed in the second chamber or removes the liquid from the second chamber through the liquid inlet,
wherein the elastic film expands towards inside the first chamber as the liquid is supplied by the liquid supply unit into the second chamber, and the elastic film is returned back to its original position as the liquid is discharged from the second chamber by the liquid supply unit.

2. The lung phantom system according to claim 1,
wherein the liquid supply unit comprises:
a liquid supply tube connected with the liquid inlet;
a liquid supplier that supplies the liquid to the liquid supply tube or takes the liquid back from the liquid supply tube; and
a driver that actuates the liquid supplier.

3. The lung phantom system according to claim 2,
wherein a driving signal of the driver is a signal having a sine waveform replicating respiration.

4. The lung phantom system according to claim 1,
further comprising a phantom that is disposed inside the first chamber, and that contracts as the elastic film expands and is returned back to its original state as the elastic film is returned.

5. The lung phantom system according to claim 4,
further comprising an air tube formed to penetrate one surface of the first chamber so as to connect the inner space and outer space of the first chamber; and
an inner film that surrounds the phantom such that the liquid does not directly contact the phantom but such that the phantom and outside air are communicable with each other through the air tube.

6. The lung phantom system according to claim 4,
wherein the phantom consists of at least two groups of blocks having different elastic moduli from each other.

7. The lung phantom system according to claim 6,
wherein the phantom consists of a plurality of layers, and a plurality of the blocks forming at least one layer of the plurality of layers consist of the at least two groups of blocks having different elastic moduli from each other.

8. The lung phantom system according to claim 6,
wherein a portion of the blocks is a target block, and when photographing the phantom with a diagnostic device, the target block is displayed differently from the other blocks.
